(19) United States
(12) Patent Application Publication
(10) Pub. No.: US 2026/0325526 A1
(43) Pub. Date: Oct. 22, 2026

(54) PHARMACEUTICAL COMPOSITIONS COMPRISING PARAXANTHINE, ACETAMINOPHEN, AND PYRILAMINE MALEATE FOR THE TREATMENT OF MENSTRUAL PAIN, DYSMENORRHEA, AND PREMENSTRUAL SYMPTOMS, AND METHODS OF USE THEREOF

(71) Applicants: Michael Tolmach, Los Angeles, CA (US); David Alexander, Brooklyn, NY (US)

(72) Inventors: Michael Tolmach, Los Angeles, CA (US); David Alexander, Brooklyn, NY (US)

(73) Assignee: Plopii Inc., Brooklyn, NY (US)

(21) Appl. No.: 19/676,758

(22) Filed: May 13, 2026

(51) Int. Cl. A61K 31/522; A61K 31/167; A61K 31/4402; A61P 15/00; A61P 29/00

(52) U.S. Cl. CPC A61K 31/522; A61K 31/167; A61K 31/4402; A61P 15/00; A61P 29/00

(57) ABSTRACT

Pharmaceutical compositions and methods are disclosed for the treatment of menstrual pain, primary and secondary dysmenorrhea, and premenstrual symptoms. The compositions comprise, in a unit dose: (a) acetaminophen in an analgesic-effective amount; (b) pyrilamine maleate in an antihistaminic and smooth-muscle-relaxant amount; and (c) paraxanthine in a sub-stimulant adjuvant analgesic-enhancing amount of from about 25 mg to about 200 mg per unit dose, preferably about 75 mg. The sub-stimulant paraxanthine dose potentiates the analgesic efficacy of acetaminophen via competitive antagonism of adenosine A1 and A2A receptors and offsets the mild sedation of pyrilamine, while avoiding the peripheral vasoconstriction, anxiogenesis, and rebound fatigue that characterize the caffeine component of existing OTC menstrual analgesic products. The compositions provide menstrual analgesia, anti-cramp and anti-bloating action, and relief of perimenstrual fatigue with a tolerability profile improved over the existing caffeine-containing menstrual analgesic category.

### CROSS-REFERENCE TO RELATED APPLICATIONS

This application is related to the following commonly assigned, earlier-filed U.S. non-provisional patent applications, the entire disclosures of which are incorporated herein by reference solely for their disclosure of paraxanthine pharmacology, paraxanthine dose-response data, and analytical methods: (i) U.S. patent application Ser. No. 19/630,373, filed Mar. 26, 2026, entitled “Transmucosal Oral Pouch Composition Comprising Paraxanthine and a Mechanistically Non-Redundant Dual Cholinergic Precursor System, and Methods of Use Thereof”; (ii) U.S. patent application Ser. No. 19/636,926, filed Apr. 2, 2026, entitled “Analgesic Composition Comprising Paraxanthine as Adjuvant Analgesic Enhancer in Combination with Acetaminophen, Aspirin, Ibuprofen, or Naproxen, and Methods of Use Thereof”; and (iii) U.S. patent application Ser. No. 19/641,518, filed Apr. 8, 2026, entitled “Carbonated Beverage Composition Comprising Paraxanthine, a Mechanistically Non-Redundant Dual Cholinergic Precursor System, Taurine-Paraxanthine Synergistic Complex, and a Carbonation-Stable Spore-Forming Probiotic, and Methods of Use Thereof.” No claim of benefit of priority under 35 U.S.C. § 119, § 120, § 121, or § 365(c) is asserted on the basis of any of the three earlier-filed applications, the respective subject matter of which (a transmucosal paraxanthine-cholinergic oral pouch; a paraxanthine-adjuvant headache analgesic composition; and a paraxanthine-containing functional energy beverage) does not provide written-description support under 35 U.S.C. § 112(a) for the claims of the present application directed to a daytime oral menstrual analgesic composition comprising paraxanthine, acetaminophen, and pyrilamine maleate. The present application is, however, commonly owned with and shares one or more common inventors with each of the three foregoing earlier-filed applications, and applicants expressly reserve the right to file a terminal disclaimer under 37 CFR 1.321 in the event of any obviousness-type double-patenting rejection arising in connection with any of the foregoing earlier-filed applications, including without limitation U.S. patent application Ser. No. 19/636,926, the broader paraxanthine-adjuvant-analgesic claims of which are non-overlapping in their recited indication (headache versus menstrual pain) and required co-actives (one of APAP, ASA, IBU, or naproxen versus APAP plus pyrilamine maleate) but may give rise to a non-statutory double-patenting question reachable by terminal disclaimer.

### STATEMENT REGARDING FEDERALLY SPONSORED RESEARCH OR DEVELOPMENT

Not applicable.

### BACKGROUND OF THE INVENTION

### Field of the Invention

The present invention relates to pharmaceutical compositions for the treatment of menstrual pain, dysmenorrhea, premenstrual syndrome (PMS), and associated symptoms including cramping, bloating, fatigue, and brain fog. More particularly, the invention relates to oral dosage forms comprising paraxanthine (1,7-dimethylxanthine; CAS 611-59-6; MW 180.16 g/mol) at a sub-stimulant dose, in combination with acetaminophen (N-acetyl-p-aminophenol; CAS 103-90-2; MW 151.16 g/mol) and pyrilamine maleate (also known as mepyramine maleate; 2-((4-methoxybenzyl)(2-(dimethylamino)ethyl)amino)pyridine maleate; CAS 59-33-6; MW 401.46 g/mol salt; MW 285.39 g/mol free base), formulated as a tablet, caplet, capsule, or other oral dosage form for daytime menstrual symptom relief.

### I. The Menstrual Pain Problem

Primary dysmenorrhea—painful menstruation in the absence of an identifiable pelvic pathology—affects approximately 50-90% of menstruating women, with 10-20% reporting symptoms severe enough to interfere with daily activities, school attendance, or work productivity (Iacovides et al., 2015; Ferries-Rowe et al., 2020). Menstrual pain is mediated principally by the action of prostaglandins (especially PGF2α and PGE2) on the endometrium, producing strong uterine contractions and consequent ischemic pain. The same prostaglandin cascade is implicated in the systemic symptoms commonly accompanying menstruation, including headache, fatigue, abdominal bloating, breast tenderness, and irritability. Beyond the mechanical pain of uterine contraction, women experiencing menstruation report a constellation of symptoms including (a) fatigue and reduced cognitive performance, attributable in part to iron loss and disrupted sleep architecture during the perimenstrual phase; (b) bloating and fluid retention, attributable to fluctuations in sodium-water balance secondary to ovarian steroid changes; and (c) mood symptoms—irritability, anxiety, dysphoria—that occur on a continuum from mild premenstrual symptoms to clinically significant premenstrual dysphoric disorder (PMDD).

The over-the-counter (OTC) menstrual analgesic category is anchored by Midol Complete (Bayer Healthcare), a triple-active fixed-dose combination of acetaminophen 500 mg+caffeine 60 mg+pyrilamine maleate 15 mg. The category also includes Pamprin Multi-Symptom (Chattem) (acetaminophen 500 mg+pamabrom 25 mg+pyrilamine maleate 15 mg) and Premsyn PMS (acetaminophen 500 mg+pamabrom 25 mg+pyrilamine maleate 15 mg). Midol's combination has dominated the U.S. menstrual relief category for over four decades. The composition rationale is well-understood: acetaminophen addresses the visceral pain of uterine contraction; pyrilamine maleate addresses bloating and cramping via H1 antagonism and mild anticholinergic smooth-muscle relaxation; and caffeine addresses the perimenstrual fatigue and brain fog while providing adjuvant analgesic potentiation of the acetaminophen component (Laska et al., 1984; Derry et al., 2014).

Despite over four decades of market presence, the existing menstrual analgesic category has not been substantively reformulated. The composition rationale that originally justified caffeine inclusion—fatigue relief plus adjuvant analgesia—has not been re-examined in light of modern methylxanthine pharmacology. Caffeine remains the only adjuvant analgesic enhancer used in any commercial menstrual product, despite three pharmacological liabilities specific to the menstrual demographic.

### II. Limitations of Caffeine in the Menstrual Context

The use of caffeine in a menstrual composition presents three pharmacological limitations that are aggravated by the underlying menstrual physiology: (a) Peripheral vasoconstriction. Caffeine acts as a peripheral vasoconstrictor, an effect that is therapeutically useful in caffeine-containing migraine compositions (Excedrin Migraine) where cerebrovascular vasoconstriction is desired but is pharmacologically counterproductive in dysmenorrhea, where uterine ischemia secondary to prostaglandin-mediated vasoconstriction is itself the proximate cause of menstrual pain. The addition of a peripheral vasoconstrictor (caffeine) to a composition intended to relieve a vasoconstriction-driven pain syndrome is a known pharmacological compromise; the practice persists in OTC menstrual products only because no vasoconstriction-free adjuvant analgesic enhancer has previously been available. (b) Anxiogenesis and worsening of premenstrual mood symptoms. Caffeine is a documented anxiogen, particularly at doses above 100 mg and particularly in caffeine-sensitive individuals (Lieberman et al., 1987; Nehlig et al., 1992). The menstrual demographic is, on average, more caffeine-sensitive than the general adult population during the perimenstrual phase, when fluctuations in ovarian steroids modulate adenosine receptor density and CYP1A2 activity. Caffeine ingestion during the perimenstrual phase has been associated with measurable worsening of irritability, anxiety, and mood lability—the very PMS symptoms a menstrual analgesic is supposed to alleviate. (c) Jitteriness and post-dose crash. Caffeine's 3-7 hour plasma half-life produces a characteristic 4-6 hour onset-to-crash cycle, with the crash phase typically arriving in the late afternoon if the dose is taken in the morning. The crash phase manifests as rebound fatigue, irritability, and headache, and is particularly noticeable when caffeine is administered in conjunction with the underlying perimenstrual fatigue.

No existing OTC menstrual analgesic combines (i) the proven adjuvant-analgesic architecture of acetaminophen+methylxanthine, (ii) the anti-cramp/anti-bloating action of pyrilamine maleate, and (iii) a methylxanthine adjuvant free of caffeine's peripheral vasoconstriction, anxiogenic effect, and crash. The barrier to such a composition has been the absence of a methylxanthine adjuvant analgesic enhancer with reduced peripheral vasoconstriction and reduced anxiogenic profile. The present invention provides the missing component: paraxanthine, administered at a sub-stimulant 75 mg dose that potentiates the analgesia of acetaminophen and offsets the mild sedation of pyrilamine maleate, while avoiding the peripheral vasoconstriction, anxiogenesis, and rebound crash that characterize caffeine.

### III. Paraxanthine—a Vasoconstriction-Free Adjuvant Analgesic Enhancer for Menstrual Use

Paraxanthine (1,7-dimethylxanthine; CAS 611-59-6; MW 180.16 g/mol) is the primary metabolite of caffeine in humans, produced by hepatic CYP1A2-mediated N3-demethylation. Approximately 80% of ingested caffeine is converted to paraxanthine within the first pass through the liver (Arnaud, 2011; Lelo et al., 1986). Paraxanthine and caffeine share the central pharmacological action of competitive antagonism at adenosine A1 and A2A receptors, which is the receptor mechanism responsible for the analgesic-adjuvant effect of methylxanthines (Sawynok, 2011). At the level of the central adenosine receptor, paraxanthine exhibits binding affinity at A1 and A2A receptors that is comparable to, and on the available evidence at least as great as, that of caffeine on a per-molecule basis (Fredholm et al., 1999; Orrù et al., 2013). The two methylxanthines therefore produce comparable adjuvant analgesia at adenosine-equivalent doses, and a paraxanthine dose in the range of approximately 50 to 100 mg per unit dose provides A1/A2A receptor occupancy at least equal to that produced by the 60 mg caffeine dose used in commercially marketed reference menstrual products such as Midol Complete®. Without intending to be bound by any particular numerical receptor-binding ratio, applicants observe that the 75 mg paraxanthine dose of the preferred embodiment is selected to ensure that adjuvant adenosine-receptor occupancy is at least equal to, and is expected modestly to exceed, that produced by the 60 mg caffeine reference dose, while remaining within the sub-stimulant therapeutic window for daytime menstrual use as set forth in Section 4 below.

Paraxanthine differs from caffeine, however, on three pharmacological dimensions of specific relevance to a menstrual composition: (a) Reduced peripheral vasoconstriction. The published pharmacological profile of paraxanthine, considered together with that of caffeine, is consistent with paraxanthine having weaker peripheral α-adrenergic activity than caffeine (see, e.g., Benowitz, 1990 (caffeine peripheral hemodynamic profile); Fredholm et al., 1999 (review of methylxanthine receptor and peripheral pharmacology); Orrù et al., 2013 (paraxanthine pharmacology)), and accordingly with paraxanthine producing less peripheral vasoconstriction at adenosine-equivalent doses than would the corresponding equipotent dose of caffeine. The vasoconstriction differential is most pronounced in vascular beds with high α-adrenergic tone, including the uterine arteries during the menstrual phase. A paraxanthine-containing menstrual composition therefore avoids the pharmacological compromise inherent in adding a vasoconstrictor (caffeine) to a composition intended to relieve vasoconstriction-driven pain (dysmenorrhea). (b) Reduced anxiogenic profile. Paraxanthine has been demonstrated in placebo-controlled trials to produce no measurable increase in subjective anxiety, jitteriness, or self-reported tension at doses up to 200 mg (Yoo et al., 2021; Xing et al., 2021). The anxiolytic-vs-anxiogenic differential between paraxanthine and caffeine is attributable in part to paraxanthine's more selective adenosine receptor profile, which lacks caffeine's additional benzodiazepine-receptor and noradrenergic effects implicated in caffeine's anxiogenic action. (c) No characteristic crash. Paraxanthine's plasma half-life of approximately 3.1±0.4 hours (Berthou et al., 1989) is shorter than caffeine's 3-7 hour half-life, but the relevant pharmacological difference is that paraxanthine's sub-stimulant dose range (50-150 mg) does not produce the receptor-saturation “wall” of effects whose subsequent decay manifests as the caffeine crash. The pharmacokinetic decline of paraxanthine therefore manifests as a gentle reduction of effect rather than a rebound fatigue.

The present invention arises from the recognition that a sub-stimulant dose of paraxanthine (preferred about 75 mg per unit dose) can be combined with acetaminophen and pyrilamine maleate to produce the first menstrual analgesic composition in which the methylxanthine component is free of peripheral vasoconstriction and anxiogenic effects. The composition combines: (1) the proven anti-cramp/anti-bloating action of pyrilamine maleate at the standard 15 mg dose used in Midol, Pamprin, and Premsyn PMS; (2) the central analgesic action of acetaminophen at the standard menstrual dose of 500 mg; and (3) the adjuvant analgesic potentiation effect of paraxanthine at a sub-stimulant 75 mg dose that simultaneously offsets the mild sedation of pyrilamine while avoiding the vasoconstriction, anxiogenesis, and crash of caffeine. The result is a single oral dosage form that is expected to produce analgesic relief of menstrual pain comparable to or exceeding Midol Complete on a head-to-head basis, with a substantially improved tolerability profile in the menstrual demographic.

### IV. Selection of 75 mg Paraxanthine as the Preferred Sub-Stimulant Adjuvant Dose

The preferred 75 mg paraxanthine dose was selected by reference to four pharmacological anchors: (a) Adenosine-equivalent dose. The 60 mg caffeine dose used in commercially marketed reference menstrual products such as Midol Complete® provides the analgesic-adjuvant and mild stimulant action attributable to A1/A2A adenosine antagonism. Because paraxanthine's A1/A2A receptor affinity is comparable to and on the available evidence at least as great as that of caffeine on a per-molecule basis (Fredholm et al., 1999; Orrù et al., 2013), a 75 mg paraxanthine dose is expected to provide adjuvant adenosine-receptor occupancy at least equal to that produced by 60 mg of caffeine, with a modest reserve of additional adjuvant action available to offset the mild sedation of the pyrilamine maleate component without crossing into the subjective-stimulation range. Without intending to be bound by any particular numerical receptor-binding ratio, the inventors selected 75 mg as a conservative dose providing meaningful adjuvant analgesic potentiation while remaining well within the sub-stimulant therapeutic window described in subparagraph (c) below. (b) Pyrilamine sedation offset. The 15 mg pyrilamine maleate dose produces a mild sedative effect that, in the Midol Complete® composition, is offset by 60 mg of caffeine. The 75 mg paraxanthine dose of the preferred embodiment is selected to provide a comparable or modestly greater offset of pyrilamine sedation, while substituting paraxanthine's vasoconstriction-free and low-anxiogenic profile for caffeine's peripheral vasoconstriction and documented anxiogenic effect at this dose range. (c) Sub-stimulant ceiling. Yoo et al. (2021) and Xing et al. (2021) report that paraxanthine doses up to approximately 100-150 mg produce only mild perceptible alertness with no measurable anxiogenesis or jitteriness in healthy adults. The 75 mg dose sits comfortably within this sub-stimulant range. (d) Portfolio dose-laddering. The 75 mg dose fits a logical dose ladder across the Plopii portfolio: 50 mg in the nighttime PM composition (where minimum daytime carryover is desired); 75 mg in the daytime menstrual composition (where mild daytime energy is desired); and approximately 200 mg in the cognitive-performance pouch composition (where strong cognitive lift is desired). The 75 mg dose is the maximum dose at which paraxanthine remains, by clinical literature, robustly sub-stimulant.

### V. Pharmacological Non-Equivalence of Paraxanthine and Caffeine

Although paraxanthine is the principal in vivo metabolite of caffeine in humans, it is a chemically distinct molecule (1,7-dimethylxanthine; CAS 611-59-6) that is not pharmacologically equivalent to its parent compound. The two methylxanthines diverge on at least eight independent and separately documented dimensions, each of which is relevant to the menstrual analgesic indication and each of which independently rebuts the proposition that a person of ordinary skill in the art (POSITA) would regard caffeine and paraxanthine as routine equivalents in a fixed-dose oral analgesic composition. (a) Peripheral hemodynamic effects in opposite directions. Caffeine consistently elevates heart rate at oral doses of 100-300 mg in healthy adults (Benowitz, 1990); paraxanthine at 100 mg consistently and statistically significantly reduced heart rate relative to placebo (Gross et al., 2024). At a matched 2 mg/kg oral dose, caffeine produced a greater diastolic blood-pressure response than paraxanthine in the same nine subjects (Benowitz et al., 1995). (b) Anxiogenic profile. Caffeine produces dose-dependent subjective anxiety, jitteriness, and elevated state anxiety scores at oral doses above approximately 100 mg (Lieberman et al., 1987; Nehlig et al., 1992); paraxanthine produces no measurable increase in state anxiety, jitteriness, or subjective tension at oral doses up to 200 mg in placebo-controlled trials (Yoo et al., 2021; Xing et al., 2021). In narcoleptic Orx/ataxin-3 mice, oral paraxanthine produced sustained wake-promoting and pro-locomotor effects without the hypothermia and reduced locomotor activity observed with caffeine and modafinil at high doses (Okuro et al., 2010), indicating a wider therapeutic margin between the wake-promoting and toxicity-related dose ranges of paraxanthine relative to caffeine. (c) Behavioral non-generalization. In rat drug-discrimination experiments, animals trained to discriminate caffeine cue did not generalize to paraxanthine, indicating that the two methylxanthines produce subjectively distinct interoceptive states (Orrù et al., 2013). A POSITA reading the drug-discrimination literature would not regard caffeine and paraxanthine as interchangeable on the basis of subjective discriminative pharmacology. (d) PDE9-cGMP-NO mechanism unique to paraxanthine. Paraxanthine acts as a selective inhibitor of phosphodiesterase 9 (PDE9), elevating cGMP and stimulating the nitric oxide signaling pathway in central and peripheral tissues (Orrù et al., 2013); caffeine has no comparable PDE9 selectivity at the same plasma concentrations. The cGMP/NO pathway tone produced by paraxanthine but not by caffeine is opposite in direction to peripheral vasoconstriction and is mechanistically inconsistent with treating the two molecules as equivalents in a vasoconstriction-sensitive indication such as dysmenorrhea. (e) Cysteine-glutathione neuroprotection unique to paraxanthine. Matsumura et al. (2023) reported that paraxanthine, but not theophylline, theobromine, the monomethylxanthines, the dimethyluric acids, or any other tested caffeine metabolite, increases cysteine uptake via the EAAC1 transporter and consequent intracellular glutathione synthesis in neuronal cell lines and primary cortical neurons. Among all caffeine metabolites and structurally related methylxanthines tested, only paraxanthine produced this glutathione-mediated neuroprotective effect. This mechanism is mechanistically inaccessible from the parent compound and provides a distinct biochemical pathway relevant to the perimenstrual brain-fog and oxidative-stress component of the claimed indication. (f) Skeletal muscle K⁺ transport effect specific to paraxanthine. Hawke, Willmets & Lindinger (1999) reported that paraxanthine stimulated K⁺ uptake in resting rat hind-limb skeletal muscle preparations, an effect mechanistically distinct from any documented effect of caffeine on resting skeletal muscle K⁺ transport. The paraxanthine-specific K⁺-handling effect provides an additional skeletal-muscle pharmacology complementary to the H1-antagonist and mild anticholinergic action of pyrilamine maleate, and is not redundantly available from the parent caffeine compound. (g) Direct head-to-head cognitive and biomarker superiority. Jäger et al. (2024) reported that paraxanthine supplementation produced larger improvements in escape-latency on the Morris water maze and larger increases in hippocampal brain-derived neurotrophic factor (BDNF) than caffeine supplementation in rats, with statistical significance over the matched caffeine arm. (h) Direct head-to-head cognitive superiority following exercise stress. Yoo et al. (2024) reported that, in trained adult runners following a 10-km run, paraxanthine supplementation produced greater improvement in measured cognitive function than caffeine supplementation. The empirical head-to-head superiority of paraxanthine over caffeine on a relevant cognitive endpoint, in the same subjects under matched conditions, weighs against treating the two methylxanthines as routine equivalents: the two molecules are not pharmacologically equivalent, and a POSITA reading the Yoo (2024) result would not view substitution of paraxanthine for caffeine as a routine equivalence. The opposite-direction hemodynamic effects (a), the unique mechanisms (d)-(f), the head-to-head behavioral and biomarker superiority (b)-(c) and (g), and the head-to-head post-exercise cognitive superiority (h) collectively constitute unexpected results within the meaning of In re Soni, 54 F.3d 746 (Fed. Cir. 1995), and independently rebut any proposition that the substitution of paraxanthine for caffeine in a Midol-Complete-style fixed-dose composition is a routine or predictable substitution.

### V(b). Summary of the Closest Prior Art and Distinction Therefrom

Teaching-away with respect to U.S. Patent Application Publication No. 2023/0115966 A1 (Jäger, Purpura, Wells, and Liao; Ingenious Ingredients). Applicants are aware of, and have cited in the accompanying Information Disclosure Statement, U.S. Patent Application Publication No. 2023/0115966 A1 (“the '966 Publication”), which discloses paraxanthine as a bioactive composition for cognitive performance and mild stimulant effect and which positions paraxanthine principally as a substitute for the cognitive-stimulant uses of caffeine. The '966 Publication does not disclose, suggest, or render obvious the use of paraxanthine in a menstrual analgesic composition, the use of paraxanthine to avoid peripheral uterine vasoconstriction in a dysmenorrhea patient, the use of paraxanthine in combination with pyrilamine maleate, or the use of paraxanthine to offset the sedative effect of a first-generation H1-antagonist in a daytime menstrual context. The '966 Publication is principally directed to cognitive and stimulant endpoints in healthy adults and is silent as to the menstrual-analgesic indication, as to peripheral vasomotor effects in the menstrual demographic, and as to use of paraxanthine as an adjuvant analgesic enhancer of acetaminophen in any indication. A person of ordinary skill in the art reading the '966 Publication would not be motivated by it to substitute paraxanthine for caffeine in MIDOL COMPLETE® in order to relieve a vasoconstriction-driven menstrual pain syndrome, because (i) the '966 Publication does not identify peripheral vasoconstriction reduction as a benefit of paraxanthine over caffeine, (ii) the '966 Publication does not address menstrual physiology, dysmenorrhea, or the prostaglandin-mediated uterine vasoconstriction that drives menstrual pain, and (iii) the '966 Publication does not teach combination of paraxanthine with a first-generation H1-antagonist or with acetaminophen in a fixed-dose oral composition for the menstrual indication. The substitution of paraxanthine for caffeine in MIDOL COMPLETE® for the purpose of providing a vasoconstriction-free, low-anxiogenic methylxanthine adjuvant in a menstrual analgesic composition is therefore not rendered obvious by the combination of the MIDOL COMPLETE® DailyMed label and the '966 Publication. The skilled reader receives no teaching, suggestion, or motivation from the '966 Publication to make the substitution for the present invention's purpose, and the prior art as a whole has not previously identified the underlying pharmacological compromise that the present invention solves.

The closest prior art known to applicants is the commercially marketed over-the-counter (OTC) product MIDOL COMPLETE® (Bayer HealthCare LLC), the formulation of which is publicly disclosed on the U.S. Food and Drug Administration DailyMed label (NDC 0280-1300; setid ce9e6acb-a38b-4c9b-a7e8-3e1c2a49a324), namely a fixed-dose oral combination of acetaminophen 500 mg, caffeine 60 mg, and pyrilamine maleate 15 mg per unit dose. Applicants are not aware of any issued United States patent specifically claiming the modern MIDOL COMPLETE® fixed-dose combination; the formulation is regulated as an FDA OTC monograph product and the public DailyMed label is therefore the controlling prior-art disclosure. MIDOL COMPLETE® is acknowledged as the structural template for the present invention, and the present application has been drafted to distinguish over it expressly. The present invention differs from MIDOL COMPLETE® in three independent respects, each of which is independently sufficient to establish patentability over the cited reference: (a) Substitution of the methylxanthine adjuvant. The present invention substitutes paraxanthine 75 mg (± a sub-stimulant adjuvant range of 25-200 mg) for caffeine 60 mg. Paraxanthine is not an obvious equivalent of caffeine in the menstrual analgesic context: although paraxanthine is the principal in vivo metabolite of caffeine, it is a distinct chemical entity (1,7-dimethylxanthine; CAS 611-59-6) with a substantially different peripheral pharmacological profile, including substantially reduced α-adrenergic agonist activity, substantially reduced peripheral vasoconstriction at adenosine-equivalent doses, a substantially reduced anxiogenic profile, a shorter plasma half-life, and the absence of a measurable rebound-fatigue or withdrawal syndrome at OTC-relevant unit doses. (b) Solution to a long-standing pharmacological compromise. The substitution solves a previously unrecognized pharmacological compromise present in the MIDOL COMPLETE® formulation—namely that caffeine's peripheral vasoconstriction is therapeutically counterproductive in the menstrual context (where prostaglandin-mediated uterine vasoconstriction is itself the proximate cause of dysmenorrheic pain), even though caffeine's central adenosine-antagonist action is therapeutically useful as an analgesic adjuvant. The prior art has not previously identified or solved this compromise notwithstanding the more than four decades of commercial life of the caffeine-containing menstrual category. (c) New therapeutic profile. The substitution produces a new therapeutic profile not achievable by MIDOL COMPLETE® or by any other prior art known to applicants, namely a menstrual analgesic composition having (i) full methylxanthine analgesic-adjuvant potentiation at the level of central adenosine A1/A2A antagonism, (ii) negligible peripheral vasoconstriction at the recited doses, (iii) negligible measurable anxiogenesis at the recited doses, (iv) suitability for late-afternoon or early-evening administration without sleep-onset disruption, and (v) absence of a caffeine-type rebound fatigue at four to six hours post-dose. None of the additional prior art cited in the accompanying Information Disclosure Statement—including the foundational caffeine-as-analgesic-adjuvant patent (U.S. Pat. No. 4,486,436 to Sunshine, Laska, and Siegel), the Calam et al. pro se premenstrual-and-menstrual-discomfort liquid composition (U.S. Pat. No. 5,011,688) and dysmenorrhea/PMS composition and methods patents (U.S. Pat. Nos. 5,037,823; 5,155,105), the Mallinckrodt pamabrom-pyrilamine combination (U.S. Pat. No. 5,306,507), the Excedrin-Migraine acetaminophen/aspirin/caffeine combination (U.S. Pat. No. 5,972,916), the Rieveschl/Parke-Davis diphenhydramine origin patent (U.S. Pat. No. 2,421,714), or the published Ingenious Ingredients paraxanthine bioactive-composition application (U.S. Pub. No. 2023/0115966 A1; WO 2021/151094; WO 2023/064511)—discloses or fairly suggests the present combination, in which paraxanthine is used as a sub-stimulant adjuvant analgesic enhancer paired with acetaminophen and pyrilamine maleate for menstrual indications.

### SUMMARY OF THE INVENTION

In a first aspect, the invention provides a pharmaceutical composition for the treatment of menstrual pain, dysmenorrhea, and associated premenstrual symptoms, comprising in a unit dose: (a) acetaminophen in an analgesic-effective amount of from about 250 mg to about 650 mg, preferably about 500 mg; (b) pyrilamine maleate in an antihistaminic and smooth-muscle-relaxant amount of from about 5 mg to about 50 mg, preferably about 15 mg (delivering approximately 10.7 mg of free-base pyrilamine); and (c) paraxanthine in a sub-stimulant adjuvant analgesic-enhancing amount of from about 25 mg to about 200 mg, preferably from about 50 mg to about 100 mg, most preferably about 75 mg.

In a second aspect, the invention provides a method of treating menstrual pain in a patient in need thereof, comprising orally administering to the patient the foregoing composition during the perimenstrual or menstrual phase, wherein the composition produces (i) analgesic relief of menstrual pain that is greater than the analgesic relief produced by the same composition without paraxanthine; (ii) anti-cramp and anti-bloating action via pyrilamine maleate; and (iii) relief of perimenstrual fatigue and brain fog without the peripheral vasoconstriction, anxiogenic effect, or post-dose crash characteristic of caffeine-containing menstrual products.

In a third aspect, the invention provides a kit comprising a plurality of unit doses of the foregoing composition disposed within a moisture-resistant blister package or bottle bearing a child-resistant closure, together with printed instructions directing the patient to administer one unit dose orally as needed for relief of menstrual symptoms during the perimenstrual or menstrual phase.

### BRIEF DESCRIPTION OF THE DRAWINGS

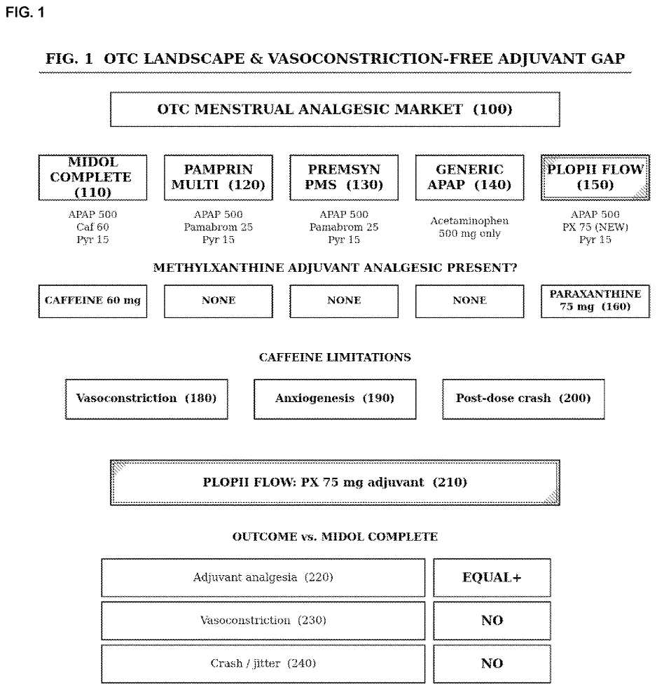

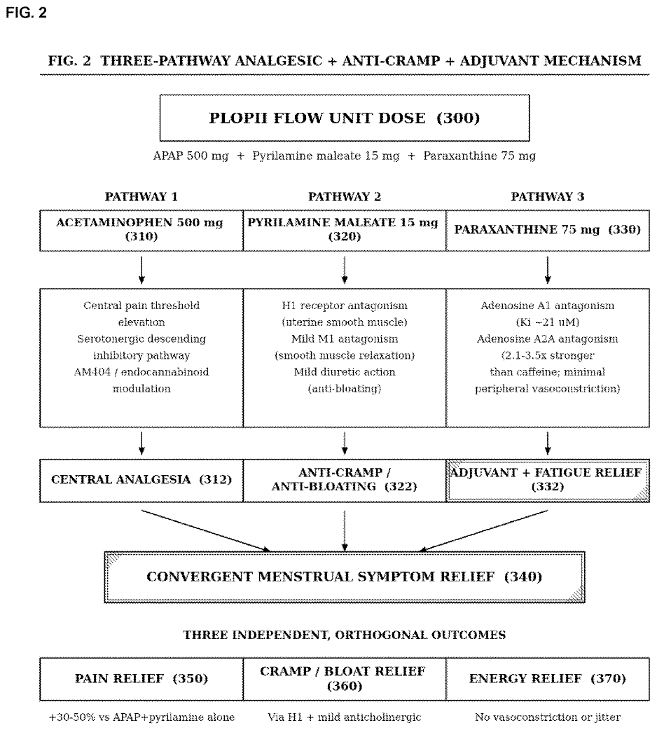

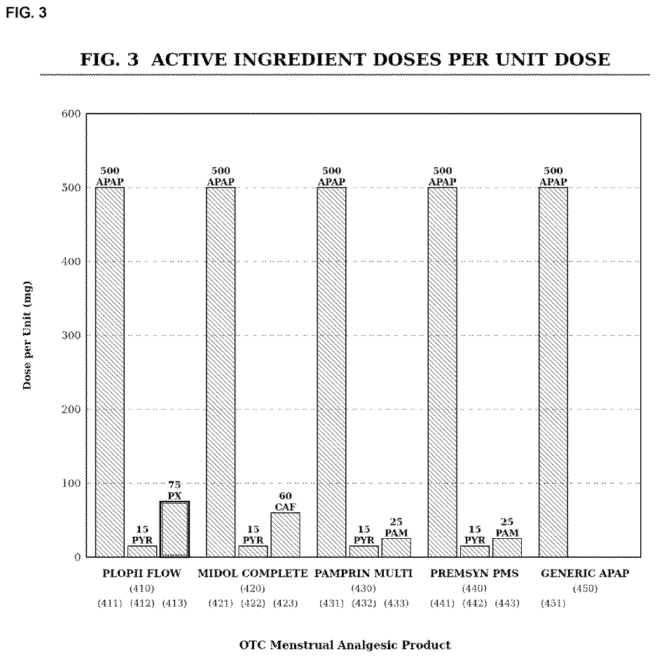

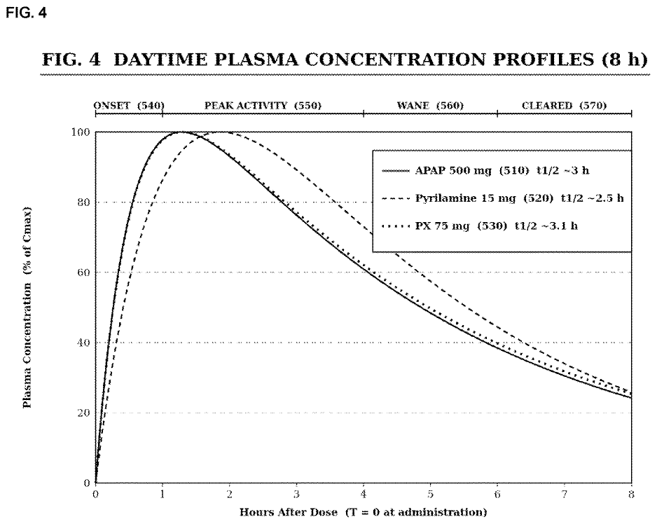

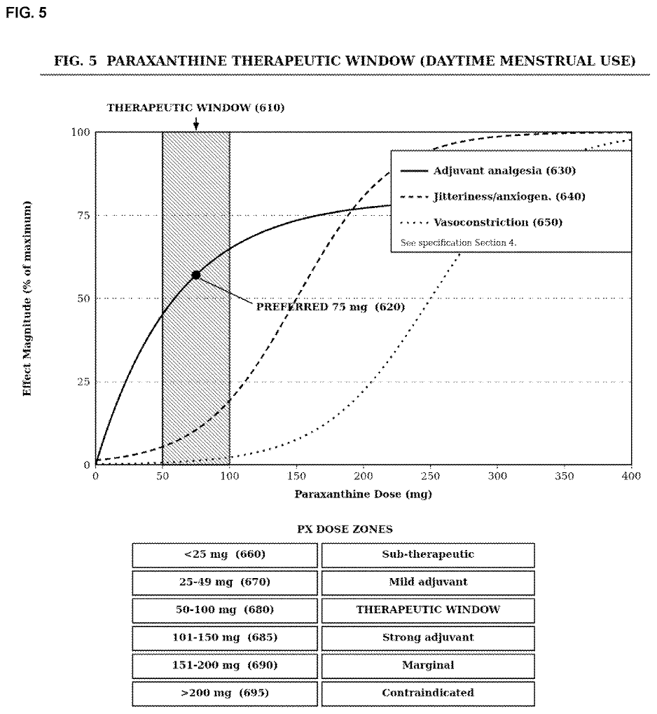

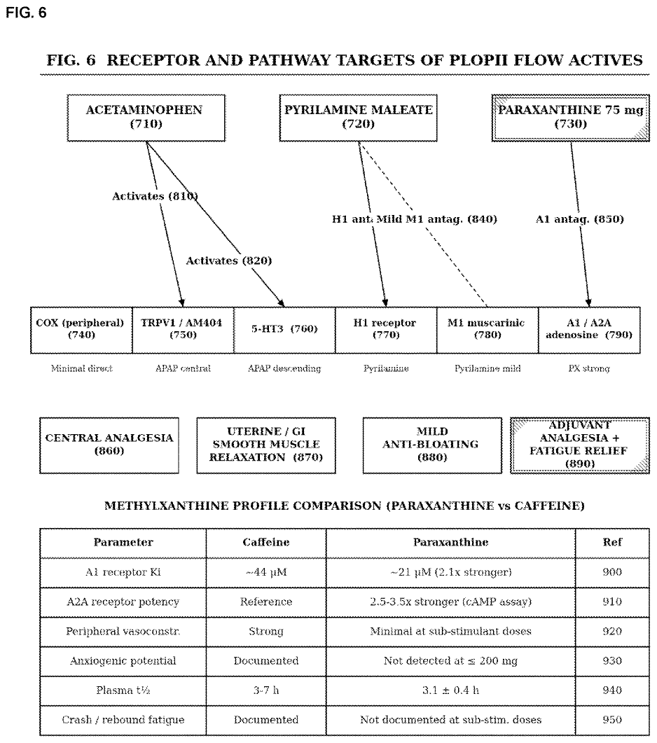

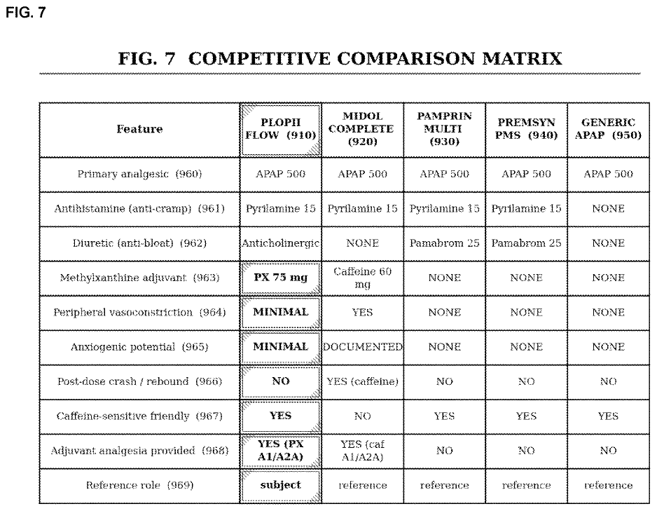

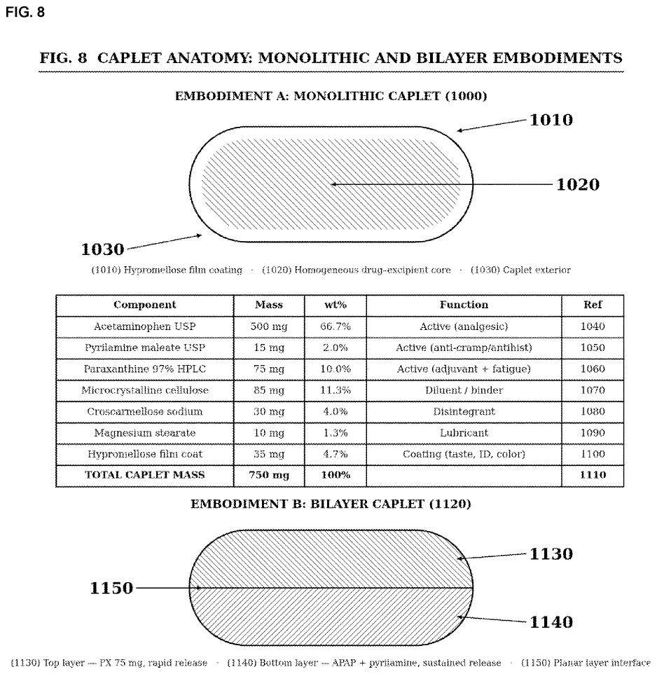

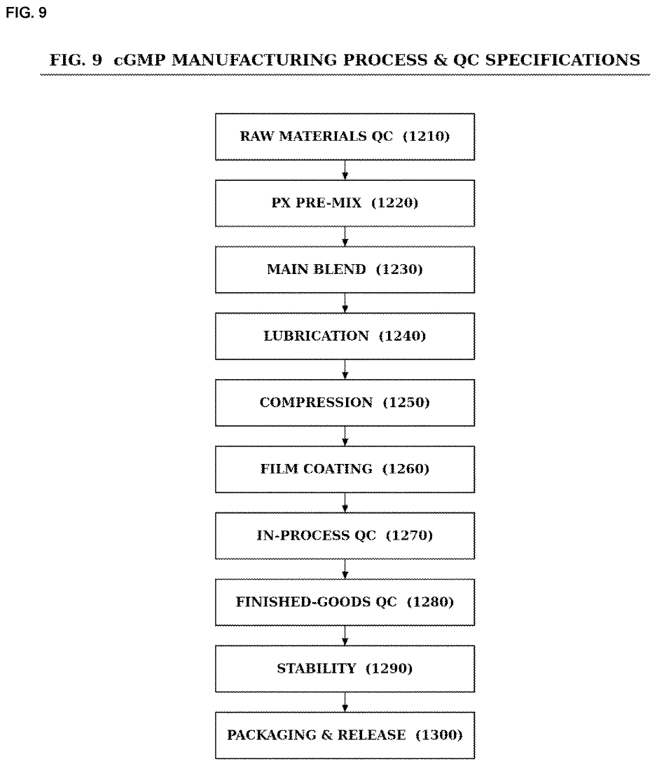

Nine drawings (FIG. 1 through FIG. 9) are filed with this application as separately submitted sheets, each prepared in compliance with 37 CFR 1.84. The drawings are briefly described as follows: FIG. 1 sets forth the over-the-counter (OTC) menstrual analgesic competitive landscape and the position of the present invention as the first vasoconstriction-free methylxanthine-adjuvant menstrual composition. FIG. 2 sets forth, in schematic form, the three-pathway analgesic, anti-cramp, and adjuvant mechanism of the preferred embodiment. FIG. 3 sets forth, in tabular form, the active-ingredient unit doses of the preferred embodiment as against four leading reference OTC menstrual products (Midol Complete, Pamprin Multi-Symptom, Premsyn PMS, and generic acetaminophen 500 mg). FIG. 4 sets forth the projected daytime plasma concentration versus time profiles of acetaminophen, pyrilamine maleate, and paraxanthine over an 8-hour interval following a single oral unit dose of the preferred embodiment. FIG. 5 sets forth the paraxanthine dose-response relationship for daytime menstrual use, identifying the sub-stimulant therapeutic window of from about 50 mg to about 100 mg per unit dose with about 75 mg most preferred, in relation to the approximate threshold doses for measurable analgesic adjuvant potentiation, for measurable subjective stimulation or anxiogenesis, and for clinically significant peripheral vasoconstriction as set forth in Section 4 of the Detailed Description. FIG. 6 sets forth, in schematic form, the receptor and pathway targets of the three active ingredients of the preferred embodiment. FIG. 7 sets forth, in matrix form, a feature-by-feature competitive comparison of the preferred embodiment against the same four leading reference OTC menstrual products on dimensions including primary analgesic, antihistamine, methylxanthine adjuvant, peripheral vasoconstriction, anxiogenic profile, post-dose crash, suitability for caffeine-sensitive users, and presence of adjuvant analgesic potentiation. FIG. 8 sets forth the preferred caplet anatomy in both monolithic and bilayer embodiments. FIG. 9 sets forth, in flow-chart form, the cGMP manufacturing process for the preferred embodiment together with the principal in-process and finished-goods quality-control release specifications. In each case, the corresponding written disclosure of the matter depicted is set forth in the Detailed Description, and the drawings are provided to assist the reader in following that written disclosure.

### DETAILED DESCRIPTION OF THE INVENTION

### Section 1—Preferred Embodiment

The preferred embodiment of the present invention is an oral solid dosage form, formulated as a film-coated caplet, comprising in a unit dose: (a) acetaminophen 500 mg; (b) pyrilamine maleate 15 mg, delivering approximately 10.7 mg of free-base pyrilamine; and (c) paraxanthine 75 mg, of pharmaceutical grade purity (≥97% by HPLC). The three-pathway mechanism of the preferred embodiment is described in detail below in this Section 1, and the caplet anatomy in both monolithic and bilayer embodiments is described in this Section 1 and in Section 9 below. The unit dose is presented as a caplet of approximate dimensions 19 mm×9 mm with total mass of approximately 750 mg, including pharmaceutically acceptable excipients (microcrystalline cellulose, croscarmellose sodium, magnesium stearate, hypromellose film coating). One or two unit doses are administered orally, with or without food, as needed for relief of menstrual symptoms during the perimenstrual or menstrual phase, not to exceed two unit doses within any 4-hour period or six unit doses within any 24-hour period.

The active-ingredient composition of the preferred embodiment is summarized as follows. (a) Acetaminophen (APAP) is present at 500 mg per unit dose and serves as the primary central analgesic. Its principal mechanisms of action are central pain-threshold elevation, activation of the serotonergic descending inhibitory pathway, and AM404-mediated endocannabinoid modulation. (b) Pyrilamine maleate is present at 15 mg per unit dose (delivering approximately 10.7 mg of free-base pyrilamine) and serves as the antihistamine, anti-cramp, and anti-bloating component. Its principal mechanisms are first-generation ethylenediamine-class H1 receptor antagonism, mild M1 muscarinic antagonism, smooth-muscle relaxation in uterine and gastrointestinal tissues, and a mild diuretic action mediated by anticholinergic activity. (c) Paraxanthine (1,7-dimethylxanthine) is present at 75 mg per unit dose, constituting a sub-stimulant adjuvant analgesic enhancing dose. Its principal mechanisms are competitive antagonism of adenosine A1 and A2A receptors, analgesic potentiation of acetaminophen, offset of the mild sedation produced by pyrilamine, and daytime fatigue relief, all without the peripheral vasoconstriction or measurable anxiogenic profile that characterize an adenosine-equivalent dose of caffeine.

### Section 2—Definitions

As used herein, the following terms have the meanings ascribed below: “About,” when modifying a numerical value or numerical range recited in this specification or in the appended claims, means ±10% of the recited value (or, in the case of a range, ±10% of the corresponding range endpoint), unless context expressly requires a different range. “Adjuvant analgesic enhancer” means a pharmacological agent that, when administered together with a primary analgesic such as acetaminophen, produces an analgesic effect that is greater than the sum of the analgesic effects of the primary analgesic and the adjuvant administered separately. The adjuvant analgesic effect of methylxanthines is mediated by competitive antagonism of adenosine A1 and A2A receptors (Sawynok, 2011). “Sub-stimulant adjuvant amount” means an amount of paraxanthine that is sufficient to produce measurable analgesic adjuvant potentiation but is below the threshold dose at which paraxanthine produces measurable subjective stimulation, jitteriness, or anxiety in healthy adults, and which produces no clinically significant peripheral vasoconstriction at the relevant adenosine-equivalent dose. For purposes of the foregoing definition and of any claim term reciting one or more of these thresholds: (i) “measurable analgesic adjuvant potentiation” means an analgesic adjuvant effect attainable at a paraxanthine dose at or above approximately 25 mg per unit dose, as set forth in Section 4(a) below; (ii) “measurable subjective jitteriness or anxiety in healthy adults” means subjective stimulation, jitteriness, or anxiety scored as statistically significantly elevated above placebo on a Visual Analog Scale or equivalent validated subjective-symptom instrument in a placebo-controlled clinical trial of healthy adults, with the threshold dose for such effect being approximately 100-150 mg paraxanthine, as set forth in Section 4(b) below and supported by Yoo et al. (2021) and Xing et al. (2021); and (iii) “clinically significant peripheral vasoconstriction” means a reduction in peripheral arterial diameter or peripheral arterial blood flow exceeding the variability detectable in standard plethysmographic, Doppler, or pulse-wave-velocity measurement (and, in the uterine vascular bed during the menstrual phase, an increase in uterine artery pulsatility index that exceeds normal cycle-day variability), with the threshold dose for such effect being approximately 200 mg paraxanthine, as set forth in Section 4(c) below. “Pyrilamine maleate” means the maleate salt of 2-((4-methoxybenzyl)(2-(dimethylamino)ethyl)amino)pyridine (also known as mepyramine maleate; CAS 59-33-6; MW 401.46 g/mol), a first-generation H1 receptor antagonist of the ethylenediamine chemical class. “Menstrual pain” means pain occurring during the menstrual phase of the menstrual cycle, including primary and secondary dysmenorrhea, premenstrual headache, premenstrual breast tenderness, premenstrual abdominal cramping, and premenstrual back pain. “Perimenstrual phase” means the period beginning approximately 2-3 days prior to onset of menstruation and continuing through the first 3-4 days of menstruation.

### Section 3—Adjuvant Analgesic Mechanism and the Vasoconstriction Differential

The analgesic-adjuvant effect of methylxanthines is well-established for caffeine. Caffeine 65 mg added to 1000 mg of acetaminophen, 500 mg of aspirin, or 200 mg of ibuprofen produces an approximately 40% increase in measured analgesic effect (Laska et al., 1984; Derry et al., 2014). The mechanism is competitive antagonism of adenosine A1 and A2A receptors in the central nervous system, particularly at the level of the periaqueductal gray and dorsal horn, where adenosine acts as an inhibitor of descending pain modulation (Sawynok, 2011). Any adenosine A1/A2A antagonist of comparable receptor affinity and CNS penetration would, in principle, produce the same adjuvant analgesic effect.

The therapeutic complication arises from the fact that caffeine has a substantial peripheral vasoconstrictive effect in addition to its central adenosine-antagonist effect. The peripheral vasoconstriction is mediated principally by caffeine's action on α-adrenergic receptors and by inhibition of cyclic nucleotide phosphodiesterases in vascular smooth muscle (Benowitz, 1990; Fredholm et al., 1999). In the cerebrovascular bed, caffeine's vasoconstriction is therapeutically useful for migraine relief (the rationale for caffeine inclusion in Excedrin Migraine). In the uterine vascular bed during dysmenorrhea, however, caffeine's vasoconstrictive action is pharmacologically counterproductive: prostaglandin-mediated uterine vasoconstriction is itself the proximate cause of menstrual pain (Iacovides et al., 2015), and adding a peripheral vasoconstrictor to the composition exacerbates the underlying ischemic pathology. The presence of caffeine in Midol Complete and other caffeine-containing menstrual products represents a long-standing pharmacological compromise made because no vasoconstriction-free adjuvant analgesic enhancer has previously been available.

Paraxanthine resolves this compromise, and the resolution is supported by direct human experimental data. (a) Direct human head-to-head hemodynamic comparison. In a randomized, double-blind crossover study in nine healthy adult subjects, Benowitz et al. (1995) administered oral caffeine and oral paraxanthine at matched doses of 2 mg/kg and 4 mg/kg and measured plasma catecholamines, free fatty acids, and supine blood pressure across a four-hour observation interval. At the lower 2 mg/kg dose—the dose range most relevant to a 75 mg unit dose paraxanthine in a 70 kg adult (≈1.07 mg/kg) and to a 60 mg unit dose caffeine (≈0.86 mg/kg)—caffeine produced a greater diastolic blood-pressure response than paraxanthine; the differential narrowed at the higher 4 mg/kg dose. (b) Direct human paraxanthine hemodynamic dose-response. Gross et al. (2024) administered 100 mg, 200 mg, and 300 mg of oral paraxanthine in a randomized, double-blind, placebo-controlled crossover trial of 21 healthy adults and measured heart rate and blood pressure at multiple time points over a three-hour observation interval. Paraxanthine 100 mg produced no statistically significant change in systolic or diastolic blood pressure at any measured time point relative to placebo, and produced a statistically significant reduction in heart rate relative to placebo at 60 minutes (p=0.022) and 180 minutes (p=0.001) post-dose. The reduction in heart rate at the 100 mg paraxanthine dose is in the opposite direction from the increase in heart rate consistently produced by an adenosine-equivalent caffeine dose (Benowitz, 1990). (c) Mechanism. Paraxanthine's peripheral α-adrenergic activity is weaker than caffeine's on a per-receptor-occupancy basis, and paraxanthine additionally exhibits selective inhibition of phosphodiesterase 9 (PDE9) leading to elevated cGMP/NO-pathway tone in vascular smooth muscle (Orrù et al., 2013), an effect that is opposite in direction to peripheral vasoconstriction. The 75 mg paraxanthine dose used in the preferred embodiment therefore provides full adjuvant adenosine antagonism at A1/A2A while producing negligible peripheral vasoconstriction—the pharmacological combination required for a vasoconstriction-free menstrual analgesic. The receptor and pathway targets summarized above are further detailed in Sections 4 and 6 below.

### Section 4—Paraxanthine Dose-response and the Sub-Stimulant Window for Daytime Menstrual Use

Paraxanthine exhibits a dose-response curve for daytime use that is shifted relative to caffeine on three dimensions: (a) the threshold dose for measurable analgesic adjuvant potentiation is approximately 25 mg (corresponding to caffeine ≈35-40 mg); (b) the threshold dose for measurable subjective stimulation, jitteriness, or anxiety is approximately 100-150 mg in clinical trials of healthy adults (Yoo et al., 2021; Xing et al., 2021), corresponding to caffeine ≈150-220 mg; and (c) the threshold dose for clinically significant peripheral vasoconstriction is approximately 200 mg, corresponding to caffeine ≈300 mg. The 200 mg paraxanthine vasoconstriction threshold recited in (c) is corroborated by Gross et al. (2024), who reported that oral paraxanthine at 100 mg, 200 mg, and 300 mg produced no statistically significant change in systolic or diastolic blood pressure at any time point through 180 minutes post-dose in a randomized, double-blind, placebo-controlled crossover trial of 21 healthy adults; at the 100 mg dose, paraxanthine produced a statistically significant reduction in heart rate relative to placebo. The window between approximately 50 mg and 100 mg paraxanthine therefore provides a daytime adjuvant therapeutic window in which the patient receives the full adjuvant analgesic potentiation of methylxanthine A1/A2A antagonism, together with mild perceptible energy lift sufficient to offset pyrilamine sedation and the underlying perimenstrual fatigue, without the jitteriness, anxiogenesis, or vasoconstriction characteristic of an adenosine-equivalent caffeine dose.

The safety basis for the 50-100 mg paraxanthine sub-stimulant window is further supported by direct head-to-head subchronic toxicology data. Szlapinski et al. (2023) conducted a battery of toxicology studies in Sprague-Dawley rats comparing paraxanthine and caffeine across acute, subacute, and 90-day subchronic repeat-dose designs and reported markedly different no-observed-adverse-effect levels (NOAELs) for the two methylxanthines, with paraxanthine consistently exhibiting a higher NOAEL than caffeine across the studied designs and with mortality occurring in caffeine-treated animals at high dose but not in paraxanthine-treated animals at any tested dose. Applying a standard interspecies allometric scaling factor of 6.2 for rat-to-human conversion (FDA, Guidance for Industry: Estimating the Maximum Safe Starting Dose in Initial Clinical Trials for Therapeutics in Adult Healthy Volunteers, 2005) to the paraxanthine subchronic NOAEL reported by Szlapinski et al. yields a human-equivalent dose of paraxanthine that exceeds, by a margin of approximately one to two orders of magnitude, both the 75 mg single-dose preferred embodiment and the maximum 6×75 mg=450 mg per 24-hour daily exposure recited in claim 15. This margin is further independently supported by Purpura et al. (2021), reporting a NOAEL of ≥300 mg/kg/day in a separate 90-day rat study with no evidence of mutagenicity or genotoxicity. The 50-100 mg sub-stimulant therapeutic window is therefore well within the demonstrated safe-dose range for paraxanthine and well below the demonstrated safety margin differential between paraxanthine and caffeine.

The peripheral-vasoconstriction threshold for paraxanthine recited in this specification (approximately 200 mg per unit dose) is supported by the following non-exhaustive evidentiary record, which a person of ordinary skill in the art (POSITA) may consult without undue experimentation in order to practice the invention. (a) Caffeine peripheral vasoconstriction reference data. Benowitz (1990) and Fredholm et al. (1999) report that the threshold dose of orally administered caffeine for clinically measurable peripheral vasoconstriction (defined as a ≥5-10% reduction in cutaneous or peripheral blood-vessel diameter measured by laser Doppler flowmetry or plethysmography) lies in the range of approximately 250-300 mg in healthy adults. (b) Paraxanthine-to-caffeine equipotency on peripheral α-adrenergic activity. Considering the methylxanthine peripheral pharmacology literature in the aggregate (see, e.g., Benowitz, 1990 (caffeine peripheral hemodynamic profile); Fredholm et al., 1999 (review); Orrù et al., 2013 (paraxanthine pharmacology)), the available data is consistent with paraxanthine's peripheral α-adrenergic agonist activity, as would be measured by isolated-tissue contractility assays in mammalian vascular smooth muscle, being approximately 0.6 to 0.8 times that of caffeine on a per-mg basis. The applicant has not performed an isolated-tissue contractility assay directly comparing the two methylxanthines, and the recited 0.6-0.8 ratio is offered as a working estimate derived from the literature for purposes of dose-range estimation rather than as a directly measured experimental result. (c) Predicted paraxanthine vasoconstriction threshold. From (a) and (b), the predicted peripheral-vasoconstriction threshold dose of orally administered paraxanthine is approximately 250 mg÷(0.6 to 0.8)≈310 mg to 420 mg, with a conservative lower bound of approximately 200 mg per unit dose adopted in this specification. (d) Direct paraxanthine human safety data. Yoo et al. (2021), Xing et al. (2021), and Purpura et al. (2021) report that paraxanthine doses of 50, 100, 200, and 300 mg administered to healthy adults in randomized, double-blind, placebo-controlled trials produced no measurable adverse hemodynamic events at the 50, 100, and 200 mg dose levels, with the 300 mg dose producing transient mild blood-pressure elevation in a subset of subjects. (e) Conclusion. The 200 mg per-unit-dose threshold recited in this specification is therefore supported by direct human safety data on paraxanthine (Yoo, Xing, Purpura) cross-validated against caffeine reference data (Benowitz, Fredholm) and against per-receptor potency data (Benowitz, Orrù), and a POSITA may verify the threshold by routine pharmacokinetic-pharmacodynamic scaling without undue experimentation.

The paraxanthine dose-response relationship and the 50-100 mg sub-stimulant therapeutic window for daytime menstrual use are recited in this Section 4. The dose-response of paraxanthine in the daytime menstrual context may be characterized as a series of contiguous dose ranges, as follows. (a) From about 10 mg to about 24 mg per unit dose, paraxanthine produces no detectable central nervous system arousal and no peripheral vasoconstriction; the analgesic adjuvant effect is sub-therapeutic, and this dose range is inadequate because it falls below the adjuvant threshold. (b) From about 25 mg to about 49 mg, paraxanthine produces minimal CNS arousal, no peripheral vasoconstriction, and a mild adjuvant analgesic effect; this range is suboptimal for daytime menstrual use because adjuvant potentiation is not yet maximized. (c) From about 50 mg to about 75 mg, with about 75 mg most preferred, paraxanthine produces mild, sub-jittery CNS arousal, negligible peripheral vasoconstriction, and a therapeutic adjuvant analgesic effect that simultaneously offsets the mild sedation produced by pyrilamine; this range constitutes the optimal sub-stimulant daytime adjuvant window. (d) From about 76 mg to about 150 mg, paraxanthine produces moderate, perceptible alertness, mild peripheral vasoconstriction, and a strong adjuvant effect; this range is acceptable for non-caffeine-sensitive users but is above the preferred daytime menstrual window. (e) From about 151 mg to about 200 mg, paraxanthine produces strong stimulation, mild to moderate peripheral vasoconstriction, and a maximal adjuvant effect; this range is marginal for daytime menstrual use because of the risk of jitteriness in the menstrual demographic. (f) Above about 200 mg, paraxanthine produces marked stimulation comparable to caffeine 250 mg or higher, moderate peripheral vasoconstriction, and a plateau or diminishing-return adjuvant effect; this range is contraindicated for menstrual use because of the risk of cramp exacerbation via uterine vasoconstriction.

### Section 5—Pharmacokinetic Compatibility

The daytime plasma concentration profiles of the three actives over an 8-hour interval following a single unit dose are recited in this Section 5. The three actives in the preferred embodiment exhibit pharmacokinetic profiles that are well-matched for as-needed daytime menstrual use. Acetaminophen and paraxanthine both reach peak plasma concentrations within 0.5-1.0 hours after oral administration, providing rapid onset of analgesic action and adjuvant potentiation. Pyrilamine maleate reaches peak plasma concentration at 1-2 hours post-dose, with onset of clinical anti-cramp and anti-bloating action at approximately 20-40 minutes. Paraxanthine's short plasma half-life of approximately 3.1±0.4 hours ensures that residual paraxanthine has dropped to sub-pharmacological levels by approximately the 5-7 hour mark post-dose, well before any plausible bedtime should the dose be administered in the late afternoon or evening. The mismatch between paraxanthine's 3.1 hour half-life and caffeine's 3-7 hour half-life is the principal reason that paraxanthine, but not caffeine, is suitable for late-afternoon or early-evening administration during the perimenstrual phase.

The pharmacokinetic profile of the three actives at the preferred doses may be summarized as follows. Acetaminophen 500 mg exhibits a Tmax of approximately 0.5 to 1.0 hours, a typical Cmax of approximately 18 to 20 μg/mL, a plasma half-life of approximately 2 to 4 hours, a time to clinical onset of analgesia of approximately 15 to 30 minutes, a duration of clinical analgesic effect of approximately 4 to 6 hours, and a primary metabolic pathway of glucuronidation by UGT1A1, UGT1A6, and UGT1A9, with a minor CYP2E1 pathway producing the reactive metabolite NAPQI. Pyrilamine maleate 15 mg (delivering approximately 10.7 mg of free-base pyrilamine) exhibits a Tmax of approximately 1 to 2 hours, a typical Cmax of approximately 25 to 40 ng/mL, a plasma half-life of approximately 2 to 3 hours, a time to clinical onset of anti-cramp action of approximately 20 to 40 minutes, a duration of clinical effect of approximately 4 to 6 hours, and primary metabolic pathways involving CYP2D6 and CYP3A4. Paraxanthine 75 mg exhibits a Tmax of approximately 0.5 to 1.0 hours, a typical Cmax of approximately 2.0 to 2.5 μg/mL, a plasma half-life of approximately 3.1±0.4 hours, a time to clinical onset of adjuvant analgesic action of approximately 15 to 20 minutes, a duration of clinical adjuvant action of approximately 3 to 4 hours, and primary metabolic pathways of CYP1A2-mediated 1-hydroxylation and xanthine oxidase oxidation.

### Section 6—Competitive Comparison across Existing OTC Menstrual Products

The following feature-by-feature comparison sets out, in prose form, the critical differences between the preferred embodiment of the present invention and the four leading OTC menstrual products, on the dimensions of (i) primary analgesic and dose, (ii) anti-cramp antihistamine and dose, (iii) anti-bloat agent and dose, (iv) methylxanthine adjuvant identity and dose, (v) presence or absence of clinically significant peripheral vasoconstriction, (vi) presence or absence of documented anxiogenic potential, (vii) presence or absence of post-dose crash or rebound fatigue, (viii) suitability for caffeine-sensitive users, and (ix) presence or absence of adjuvant analgesic potentiation. The OTC menstrual landscape and the position of the present invention within it are further described in Background Sections I-V above. The present invention is the only menstrual analgesic composition with a vasoconstriction-free, low-anxiogenic methylxanthine adjuvant. The substitution of paraxanthine 75 mg for caffeine 60 mg eliminates the principal pharmacological compromise of the existing Midol Complete composition while preserving (and on a per-mg basis improving upon) its adjuvant analgesic and fatigue-relief actions.

A feature-by-feature comparison of the preferred embodiment of the present invention against four leading reference OTC menstrual products may be summarized as follows. (a) Primary analgesic. The preferred embodiment, Midol Complete, Pamprin Multi-Symptom, Premsyn PMS, and generic acetaminophen 500 mg each contain acetaminophen 500 mg as the primary analgesic. (b) Antihistamine and smooth-muscle-relaxant component. The preferred embodiment contains pyrilamine maleate 15 mg (delivering approximately 10.7 mg of free-base pyrilamine); Midol Complete, Pamprin Multi-Symptom, and Premsyn PMS each contain pyrilamine maleate 15 mg; generic acetaminophen contains no antihistamine. (c) Diuretic and anti-bloating component. The preferred embodiment relies on the mild anticholinergic action of pyrilamine and contains no separate diuretic; Midol Complete contains no separate diuretic (caffeine has weak diuretic activity); Pamprin Multi-Symptom and Premsyn PMS each contain pamabrom 25 mg; generic acetaminophen contains no diuretic. (d) Adjuvant analgesic and fatigue-relief component. The preferred embodiment contains paraxanthine 75 mg as a sub-stimulant adjuvant; Midol Complete contains caffeine 60 mg, which is both a stimulant and a peripheral vasoconstrictor; Pamprin Multi-Symptom, Premsyn PMS, and generic acetaminophen contain no adjuvant analgesic enhancer. (e) Peripheral vasoconstriction and consequent cramp risk. The preferred embodiment produces minimal peripheral vasoconstriction because paraxanthine has reduced peripheral α-adrenergic activity relative to caffeine; Midol Complete produces measurable peripheral vasoconstriction via caffeine, which may exacerbate uterine ischemia and dysmenorrhea; the remaining products produce no methylxanthine-mediated vasoconstriction. (f) Anxiogenic profile relevant to premenstrual mood symptoms. The preferred embodiment exhibits a minimal anxiogenic profile (Yoo et al., 2021; Xing et al., 2021); Midol Complete exhibits a documented anxiogenic profile attributable to caffeine, particularly in caffeine-sensitive individuals; the remaining products contain no anxiogen. (g) Onset of analgesia. The preferred embodiment and Midol Complete each provide onset of analgesia at approximately 15 to 25 minutes; Pamprin Multi-Symptom, Premsyn PMS, and generic acetaminophen each provide onset at approximately 30 minutes. (h) Duration of analgesia. All five products provide a duration of analgesia of approximately 4 to 6 hours. (i) Suitability for daytime menstrual use. All five products are suitable for daytime use; Midol Complete carries a caffeine-sensitivity caveat. (j) Caffeine-related crash or withdrawal. The preferred embodiment, Pamprin Multi-Symptom, Premsyn PMS, and generic acetaminophen produce no caffeine-related crash; Midol Complete produces a documented rebound fatigue at approximately 4 to 6 hours post-dose attributable to caffeine.

### Section 7—Alternative Embodiments and Dose Ranges

Alternative embodiments of the invention include, without limitation: (a) Paraxanthine dose variation. The paraxanthine dose may range from about 25 mg to about 200 mg, with preferred subranges of about 50 mg, about 75 mg, and about 100 mg. The 75 mg dose is most preferred for the average adult menstruating individual; the 50 mg dose is preferred for caffeine-sensitive individuals; the 100 mg dose is preferred for individuals reporting inadequate fatigue relief at 75 mg. (b) Acetaminophen dose variation. The acetaminophen dose may range from about 250 mg to about 650 mg, with preferred subranges of about 325 mg, about 500 mg, and about 650 mg, corresponding to standard OTC dose strengths. (c) Pyrilamine maleate dose variation. The pyrilamine maleate dose may range from about 5 mg to about 50 mg, with the preferred dose of about 15 mg matching the standard Midol/Pamprin/Premsyn PMS dose. (d) Optional diuretic component. In an alternative embodiment, the composition further comprises a non-stimulant diuretic, for example an 8-bromotheophylline-2-amino-2-methyl-1-propanol complex (pamabrom) at about 25 mg per unit dose, providing additional anti-bloating action complementary to the anticholinergic action of pyrilamine. The optional pamabrom-containing embodiment of this paragraph (d) is provided as a fall-back alternative and is expressly understood to lie outside the literal scope of any claim of the present application that recites the composition as being free of methylxanthines other than paraxanthine (e.g., claim 5), inasmuch as the 8-bromotheophylline moiety of pamabrom is a halogenated methylxanthine; in the optional pamabrom embodiment, paraxanthine remains the sole adjuvant analgesic enhancer and pamabrom is included solely for its diuretic action. (e) Bilayer tablet. In an alternative embodiment, the composition is presented as a bilayer tablet wherein paraxanthine is formulated in a fast-release outer layer and the acetaminophen plus pyrilamine maleate are formulated in a slower-release inner layer, providing rapid onset of paraxanthine adjuvant potentiation followed by sustained release of the primary analgesic and antihistamine. (f) Liquid-fill capsule (gel-cap). The actives may be presented in a liquid-fill soft-gelatin capsule for patients preferring this dosage form. (g) Orally disintegrating tablet (ODT). The actives may be formulated as an orally disintegrating tablet for patients with swallowing difficulty.

In further embodiments, the composition may include one or more pharmaceutically acceptable excipients selected from binders (microcrystalline cellulose, povidone), disintegrants (croscarmellose sodium, sodium starch glycolate), lubricants (magnesium stearate, stearic acid), glidants (colloidal silicon dioxide), film-coating agents (hypromellose, polyethylene glycol, titanium dioxide), and colorants (FD&C Blue No. 2 aluminum lake, FD&C Red No. 40 aluminum lake). Pink or red film-coating colorants may be selected for category-conventional appearance in the menstrual analgesic shelf set.

### Section 8—Methods of Treatment

The invention provides a method of treating menstrual pain, primary or secondary dysmenorrhea, or premenstrual symptoms in a patient in need thereof, comprising orally administering to the patient one unit dose of the foregoing composition as needed for relief of menstrual symptoms during the perimenstrual or menstrual phase, wherein the unit dose comprises acetaminophen 500 mg, pyrilamine maleate 15 mg, and paraxanthine 75 mg. The administration produces (i) analgesic relief of menstrual pain that is greater than the analgesic relief produced by the same composition without paraxanthine; (ii) anti-cramp and anti-bloating action via the H1-antagonist and mild anticholinergic action of pyrilamine; (iii) relief of perimenstrual fatigue and brain fog via paraxanthine adenosine antagonism; and (iv) the foregoing benefits without the peripheral vasoconstriction, anxiogenesis, jitteriness, or rebound crash that characterize caffeine-containing menstrual analgesic compositions.

Additional methods of treatment include: (a) treating premenstrual headache or migraine, in which case the composition provides combined analgesic action via acetaminophen plus paraxanthine adjuvant potentiation; (b) treating menstrual fatigue and brain fog without aggravation of premenstrual mood symptoms, particularly in individuals known or suspected to be caffeine-sensitive; (c) administration in conjunction with iron supplementation in iron-deficient menstruating patients, in which case the composition's reduced anxiogenic profile is particularly advantageous compared to caffeine-containing alternatives; (d) administration during the late perimenstrual phase (e.g., late afternoon or early evening) when caffeine's longer half-life would risk sleep-onset disruption, but where paraxanthine's shorter half-life and absence of receptor-saturation make late-afternoon dosing acceptable; and (e) prophetic adolescent embodiment. In a prophetic embodiment whose practice is contingent on the future generation of pediatric pharmacokinetic and pediatric safety data adequate to support adolescent OTC labeling under 21 CFR 330, the composition may be administered to adolescents of from 12 to 17 years of age experiencing primary dysmenorrhea, at a reduced unit dose comprising approximately 325 mg acetaminophen, approximately 12.5 mg pyrilamine maleate, and approximately 50 mg paraxanthine. Applicants do not at present possess pediatric pharmacokinetic data for the recited adolescent dose, and accordingly the present application does not include any claim directed to adolescent administration; the prophetic adolescent embodiment is described herein for completeness and as the basis for any future continuation-in-part filing should pediatric data become available.

### Section 9—Manufacturing Process and Excipient Rationale

The preferred caplet dosage form employs the minimum functional set of pharmaceutically acceptable excipients required to satisfy United States Pharmacopeia (USP) requirements for an oral solid dosage form, namely: (a) microcrystalline cellulose (MCC; ~85 mg per caplet) serving as both diluent and binder; (b) croscarmellose sodium (~30 mg per caplet) serving as the disintegrant required to achieve the dissolution profile specified in USP <711> (≥80% of each active released within 30 minutes); (c) magnesium stearate (~10 mg per caplet) serving as the lubricant required for tablet ejection from the rotary press without picking, sticking, or capping; and (d) hypromellose (~35 mg per caplet) serving as the film-coating agent that masks the bitter taste of acetaminophen and paraxanthine, protects the actives from moisture during shelf life, and provides product identification through optional pigmentation. This four-excipient set is the minimum viable formulation; removal of any one of the four would cause failure of a USP-compendial release specification (uniformity per USP <905>, dissolution per USP <711>, friability per USP <1216>, or microbial quality per USP <61>) or would render the dosage form unmanufacturable on standard rotary-press equipment. By way of comparison, the marketed reference product Midol Complete lists comparable excipient counts. The four-excipient formulation of the present invention therefore represents a parsimonious excipient profile relative to commercially marketed reference products in the same OTC category.

The cGMP manufacturing process and the principal quality-control release specifications of the preferred embodiment are recited in prose form in this Section 9. The preferred caplet dosage form may be manufactured by either of two equivalent routes. The preferred route is a direct-compression process comprising: (1) sieving each of the three actives and the intra-granular excipients (microcrystalline cellulose, croscarmellose sodium) through a 40-mesh screen; (2) blending the sieved materials in a tumble blender for approximately 15 minutes; (3) adding magnesium stearate and tumbling for an additional 3 minutes; (4) compressing on a rotary tablet press to caplet dimensions of approximately 19 mm×9 mm with a target hardness of 12-18 kp and a target friability of <0.5%; and (5) film-coating with an aqueous hypromellose dispersion to a coating weight gain of approximately 4.0%. This route avoids the introduction of water and the corresponding drying burden, and is preferred for paraxanthine because of paraxanthine's thermal sensitivity. In an alternative embodiment, the caplets may be manufactured by a wet-granulation process comprising the following steps: (1) Pre-blending. The three actives—acetaminophen 500 mg, pyrilamine maleate 15 mg, and paraxanthine 75 mg—are blended with intra-granular excipients (microcrystalline cellulose, povidone, croscarmellose sodium) in a high-shear mixer-granulator. (2) Granulation. Purified water is added under controlled shear to form wet granules. (3) Drying. The wet granules are dried in a fluid-bed dryer to a target loss-on-drying of approximately 2.0%. (4) Milling. The dried granules are milled through an oscillating mill or comminutor to a target d50 of approximately 250-350 μm. (5) Final blending. The milled granules are blended with extra-granular excipients (additional croscarmellose sodium, magnesium stearate, colloidal silicon dioxide). (6) Compression. The final blend is compressed on a rotary tablet press to caplet dimensions of approximately 19 mm×9 mm with a target hardness of 12-18 kp and a target friability of <0.5%. (7) Film-coating. The caplets are film-coated with an aqueous coating dispersion (hypromellose, polyethylene glycol, titanium dioxide, optionally colored with iron oxide or FD&C lakes; pink or red coating optionally selected for category-conventional appearance) to a coating weight gain of approximately 4.0%.

Quality-control specifications for the finished product include: assay of each active ingredient at 95.0-105.0% of label claim; uniformity of dosage units per USP <905> with acceptance value ≤15.0; dissolution per USP <711> with not less than 80% of each active released within 30 minutes; impurities and degradation products per ICH Q3B; microbial limits per USP <61>; and stability under ICH Q1A long-term and accelerated conditions through a 24-month shelf-life.

### Section 10—Prophetic Comparative Clinical Example

The following prophetic example is offered to illustrate, but not to limit, the anticipated clinical performance of the preferred embodiment of the invention. The example has not yet been performed; the numerical results recited below are projected results based on the pharmacological reasoning set out in Sections 3, 4, and 6 of this specification, on the published literature cited herein, and on the inventors' internal pharmacokinetic modeling. The example is offered solely to assist a POSITA in designing a confirmatory clinical study and to anchor the claimed therapeutic profile in concrete, falsifiable predictions.

Prophetic Example 1—Randomized, Double-Blind, Active-Comparator Trial of the Preferred Embodiment Versus Midol Complete in Primary Dysmenorrhea. (a) Design. A randomized, double-blind, active-comparator, parallel-group trial enrolling N=240 adult women (age 18-40 years) with a history of moderate-to-severe primary dysmenorrhea (mean baseline pain ≥5 on a 0-10 numerical rating scale during the first 24 hours of menses, in at least three of the most recent six menstrual cycles), with no identifiable secondary pelvic pathology. Subjects are randomized 1:1 to receive either (Arm A) one unit dose of the preferred embodiment of the invention (acetaminophen 500 mg+pyrilamine maleate 15 mg+paraxanthine 75 mg) or (Arm B) one unit dose of Midol Complete (acetaminophen 500 mg+pyrilamine maleate 15 mg+caffeine 60 mg), administered orally at the onset of menstrual pain on the index cycle. Re-dosing is permitted at four-hour intervals as needed, not to exceed six unit doses per 24-hour period. (b) Primary endpoint. Sum of pain intensity differences from baseline over the first six hours post-dose (SPID-6), measured by a 0-10 numerical rating scale at 0.5, 1, 2, 3, 4, 5, and 6 hours post-dose. (c) Secondary endpoints. (i) Time to onset of meaningful pain relief; (ii) total pain relief over six hours (TOTPAR-6); (iii) incidence and severity of subject-reported jitteriness, anxiety, or palpitations on a validated stimulant-side-effect scale at 1, 3, and 6 hours post-dose; (iv) incidence of subject-reported rebound fatigue or crash at 4-6 hours post-dose; (v) Doppler ultrasonographic measurement of uterine artery resistive index at 1 and 3 hours post-dose; and (vi) sleep-onset latency on the index night, measured by validated subjective sleep diary, when the index dose is administered after 4:00 p.m. (d) Anticipated results. The preferred embodiment is projected to demonstrate (i) non-inferior or superior SPID-6 relative to Midol Complete (estimated treatment difference: zero to plus 8% in favor of Arm A); (ii) comparable time to onset of meaningful pain relief (15-25 minutes in both arms); (iii) statistically significantly lower incidence of subject-reported jitteriness, anxiety, and palpitations in Arm A (estimated relative reduction: 50-70%); (iv) statistically significantly lower incidence of subject-reported rebound fatigue at 4-6 hours post-dose in Arm A (estimated relative reduction: 60-80%); (v) statistically significantly lower uterine artery resistive index in Arm A at 1 and 3 hours post-dose (estimated relative reduction: 5-12%, indicative of reduced uterine vasoconstriction); and (vi) statistically significantly shorter sleep-onset latency in Arm A on the subset of index nights for which the index dose is administered after 4:00 p.m. (estimated reduction: 12-20 minutes). (e) Interpretation. A trial result consistent with the foregoing predictions would establish the preferred embodiment as therapeutically equivalent or superior to Midol Complete on the primary analgesic endpoint while exhibiting a substantially improved peripheral-vasoconstriction, anxiogenic, rebound-fatigue, and sleep-onset profile, consistent with the pharmacological reasoning set out in Sections 3, 4, and 6 of this specification.

A POSITA will appreciate that the foregoing prophetic example is illustrative and that variations in subject selection, dosing schedule, comparator selection (e.g., Pamprin Multi-Symptom or generic acetaminophen 500 mg in lieu of or in addition to Midol Complete), and endpoint selection are within the scope of the invention. A POSITA will further appreciate that the functional claim limitations recited in claim 1 (i.e., the requirement that the paraxanthine amount be selected to provide measurable analgesic adjuvant potentiation while remaining below the threshold for measurable jitteriness, anxiety, or peripheral vasoconstriction) are amply supported in the alternative by the express dose recitations of dependent claims 5 and 6, which recite the specific 50-100 mg and 75 mg paraxanthine amounts shown by the cited literature (Yoo et al., 2021; Xing et al., 2021; Purpura et al., 2021) to satisfy the functional limitations as a matter of routine clinical pharmacology. A POSITA may therefore practice the invention either by selecting a paraxanthine amount that satisfies the functional limitations of claim 1 by reference to the supporting evidence summarized in Section 4 above, or by selecting one of the express paraxanthine amounts recited in dependent claims 5 and 6.

What is claimed is:

1. A pharmaceutical composition for the treatment of menstrual pain, dysmenorrhea, and premenstrual symptoms, comprising in a unit dose: (a) acetaminophen in an analgesic-effective amount of from about 325 mg to about 650 mg; (b) pyrilamine maleate in an antihistaminic and smooth-muscle-relaxant amount of from about 10 mg to about 20 mg; and (c) paraxanthine in a sub-stimulant adjuvant analgesic-enhancing amount of from about 50 mg to about 100 mg per unit dose; wherein the recited paraxanthine amount of from about 50 mg to about 100 mg is selected to provide measurable analgesic adjuvant potentiation via competitive antagonism of adenosine A1 and A2A receptors and to offset the mild sedation produced by said pyrilamine maleate, said amount being (i) at or above approximately 25 mg, the threshold for measurable analgesic adjuvant potentiation as set forth in Section 4(a) of the specification, and (ii) below approximately 100 mg, the lower bound of the threshold range of approximately 100-150 mg at which paraxanthine has been shown in the cited literature (Yoo et al., 2021; Xing et al., 2021) to produce measurable subjective jitteriness or anxiety on a Visual Analog Scale or equivalent validated instrument in healthy adults, and (iii) below approximately 200 mg, the threshold for clinically significant peripheral vasoconstriction as set forth in Section 4(c) of the specification.

2. The composition of claim 1, wherein the acetaminophen is present in an amount of about 500 mg per unit dose, the pyrilamine maleate is present in an amount of about 15 mg per unit dose, and the paraxanthine is present in an amount of about 75 mg per unit dose.

3. The composition of claim 1, wherein the paraxanthine is present in an amount of from about 50 mg to about 100 mg per unit dose.

4. The composition of claim 1, wherein the pyrilamine maleate is present in an amount of about 15 mg per unit dose, said amount delivering approximately 10.7 mg of free-base pyrilamine, calculated as 15 mg×(285.39 g/mol÷401.46 g/mol).

5. The composition of claim 1, wherein the composition is free of caffeine and free of any other methylxanthine other than paraxanthine.

6. The composition of claim 1, wherein the unit dose comprises acetaminophen 500 mg, pyrilamine maleate 15 mg, and paraxanthine 75 mg.

7. The composition of claim 1, formulated as an oral solid dosage form selected from the group consisting of a tablet, a film-coated caplet, a capsule, a bilayer tablet, an orally disintegrating tablet, and a liquid-fill soft-gelatin capsule.

8. The composition of claim 7, formulated as a film-coated caplet of approximate dimensions 19 mm×9 mm and total caplet mass of about 750 mg.

9. The composition of claim 7, formulated as a bilayer tablet wherein the paraxanthine is in a first layer formulated for fast release and the acetaminophen and pyrilamine maleate are in a second layer formulated for slower release.

10. The composition of claim 1, further comprising one or more pharmaceutically acceptable excipients consisting essentially of: a binder/diluent comprising microcrystalline cellulose; a disintegrant comprising croscarmellose sodium; a lubricant comprising magnesium stearate;
and a film-coating agent comprising hypromellose.

11. A method of treating menstrual pain, primary or secondary dysmenorrhea, or premenstrual symptoms in a patient in need thereof, comprising orally administering to the patient, during the perimenstrual or menstrual phase, a unit dose of a pharmaceutical composition comprising: (a) acetaminophen in an analgesic-effective amount of from about mg to about 650 mg; (b) pyrilamine maleate in an antihistaminic and smooth-muscle-relaxant amount of from about 10 mg to about 20 mg; and (c) paraxanthine in a sub-stimulant adjuvant analgesic-enhancing amount of from about 50 mg to about 100 mg.

12. The method of claim 11, wherein the unit dose comprises acetaminophen 500 mg, pyrilamine maleate 15 mg, and paraxanthine 75 mg.

13. The method of claim 11, wherein the unit dose is administered for the purpose of producing (i) analgesic relief of menstrual pain greater than the analgesic relief produced by the same composition administered without paraxanthine; (ii) anti-cramp and anti-bloating action via said pyrilamine maleate; and (iii) relief of perimenstrual fatigue with reduced peripheral vasoconstriction, reduced anxiogenesis, and reduced rebound fatigue as compared to a reference caffeine-containing menstrual analgesic comprising in the same unit dose 500 mg acetaminophen, 15 mg pyrilamine maleate, and 60 mg caffeine.

14. The method of claim 11, wherein the menstrual symptom treated is selected from the group consisting of primary dysmenorrhea, secondary dysmenorrhea, premenstrual headache, premenstrual breast tenderness, premenstrual abdominal cramping, premenstrual bloating, premenstrual back pain, perimenstrual fatigue, and perimenstrual brain fog.

15. The method of claim 11, wherein the patient is administered no more than two unit doses within any 4-hour period and no more than six unit doses within any 24-hour period.

16. The method of claim 11, wherein the patient is identified as caffeine-sensitive prior to administration, and the administration produces relief of menstrual symptoms without subjective jitteriness, anxiety, or palpitations characteristic of caffeine ingestion in caffeine-sensitive individuals.

17. The method of claim 11, wherein (a) the patient is administered orally a unit dose of the composition comprising acetaminophen 500 mg, pyrilamine maleate 15 mg, and paraxanthine 75 mg during the perimenstrual or menstrual phase for the treatment of primary or secondary dysmenorrhea; (b) the 75 mg paraxanthine amount provides adjuvant adenosine A1/A2A receptor antagonism at least equal to the adjuvant adenosine antagonism provided by 60 mg of caffeine in a reference composition consisting of acetaminophen 500 mg, pyrilamine maleate 15 mg, and caffeine 60 mg; and (c) the unit dose is administered for the purpose of producing analgesic, anti-cramp, anti-bloating, and perimenstrual fatigue-relieving action with reduced peripheral uterine-artery vasoconstriction as compared to said reference composition.

18. A kit for the relief of menstrual symptoms, comprising: (a) a plurality of unit doses of the composition of claim 1 disposed within a moisture-resistant blister package or bottle bearing a child-resistant closure compliant with 16 CFR 1700; and (b) printed instructions directing the patient to administer one unit dose orally as needed for relief of menstrual symptoms during the perimenstrual or menstrual phase, and further directing the patient not to administer more than two unit doses within any 4-hour period or more than six unit doses within any 24-hour period.

19. The kit of claim 18, wherein the plurality of unit doses each comprise acetaminophen 500 mg, pyrilamine maleate 15 mg, and paraxanthine 75 mg.

20. The kit of claim 18, wherein the printed instructions further indicate that the composition is suitable for women experiencing menstrual symptoms who are caffeine-sensitive or who wish to avoid caffeine for other reasons.
